(19) United States
(12) Patent Application Publication
(10) Pub. No.: US 2023/0127507 A1
(43) Pub. Date: Apr. 27, 2023

(54) INTRACRANIAL ABNORMALITY REMOVAL SYSTEM

(71) Applicant: Ninh B. Doan, Fairhope, AL (US)

(72) Inventor: Ninh B. Doan, Fairhope, AL (US)

(21) Appl. No.: 17/991,984

(22) Filed: Nov. 22, 2022

Related U.S. Application Data
(63) Continuation of application No. PCT/US21/56472, filed on Oct. 25, 2021.

(51) Int. Cl. A61B 17/34; A61B 17/00

(52) U.S. Cl. CPC A61B 17/3421; A61B 17/00234; A61B 2017/22079

(57) ABSTRACT

A surgical system for removal of an intracranial abnormality of predetermined size and position. The system includes an elongated suction tube including an interior passage extending between an open proximal end and an open distal end. The suction tube includes an elongated main body portion disposed between the open distal end and a forward end of an enhanced diameter collection cavity. The cross-sectional area of the interior passage varies along the length of the suction tube and is greater within the collection cavity than within the main body portion. The total internal volume of the collection cavity may exceed the volume necessary to contain the abnormality.

### TECHNICAL FIELD

The present disclosure relates generally to medical devices, and more particularly to a system adapted for insertion into the brain of a human or other mammal for vacuum extraction of cerebral hematomas or other abnormalities such as abscesses, glioblastomas, metastases and the like.

### BACKGROUND

The brain is made up of soft tissue containing a complex and sensitive neural network connected to the body through the spinal cord. The brain is protected by the surrounding skull making access to abnormalities such as intracranial cerebral hematomas, abscesses, glioblastomas, metastases and the like a highly complex and difficult procedure. Moreover, to gain access to a hematoma or other abnormality for removal, it is generally necessary to pass through surrounding brain tissue. To avoid damaging the delicate neural network, it is generally desirable to minimize disruption of the surrounding brain tissue while maneuvering to the location of interest.

It is generally known that intracranial cerebral hematomas and other abnormalities may be treated by removal using tubular suction devices. However, to avoid undue brain tissue trauma, practitioners have generally relied on relatively narrow diameter elongated devices to minimize undue disruption of the surrounding brain tissue during insertion. One such system is illustrated and described in U.S. Pat. No. 10,022,520 which is incorporated herein by reference in its entirety. While devices with narrow internal diameters may provide the benefit of minimizing disruption to surrounding tissue, the narrow interior diameter of such devices may make the complete removal of the hematoma or other abnormality more difficult due to clogging during the procedure. Thus, the desired removal procedure may be more complex to carry out and/or complete removal may not be possible. These difficulties may be particularly acute in instances where the hematoma is non-superficial and is located within the so called “white matter” of the brain where access is more difficult and substantial instrument manipulation may be undesirable.

When using traditional suction devices, it is known to use imaging technologies such as stereotactic X-ray imaging, Computerized Axial Tomography (CAT scans), Computerized Tomographic Angiography, Position Emission Tomography (PET scans), Magnetic Resonance Imaging (MRI) and the like to identify the location of the hematoma or other abnormality intended for removal. The resulting images essentially provide a map for access to the location of interest. In one common practice, a fine diameter positioning probe such as a Medtronic Stealth Probe or the like may then be linked to the scan and can be positioned at the interior of the suction tube to help the physician guide the tube into position prior to activating the suction function. This positioning operation ensures precise placement of the suctioning force. However, the use of a suction tube with a narrow effective internal diameter to avoid tissue disruption during insertion may have the undesired consequence of making complete removal of the hematoma or other abnormality unduly difficult.

Simply increasing the suction tube diameter may not be a desirable solution. In this regard, while known systems may provide a preferred pathway and positioning guide for a hematoma removal suctioning device, the insertion of the suction tube nonetheless creates a potential issue since brain tissue will necessarily be impacted to some degree by the initial insertion. Larger diameter suction tubes may increase undesired trauma during insertion. To control any potential insertion trauma, prior systems have often required a significant degree of dissection to establish a desired pathway for the suction tube. However, even with an established pathway, the permissible diameter of the suction tube may still be substantially limited to avoid undue matter displacement during the insertion procedure.

Finally, even when current suction-based systems have been properly inserted and positioned, the narrow effective internal diameter of current suction tubes may necessitate the application of an unduly high suctioning force to extract the abnormality and withdraw the matter through the narrow interior channel and into an attached suctioning line. Using excessively high suctioning forces through a narrow extraction tube may also be problematic. Specifically, as the applied suctioning force is increased, there is a corresponding potential for potentially undesirable spikes in the applied force experienced by the healthy tissue surrounding the hematoma. That is, as removed material enters the suction tube, there will necessarily be some reduction in the suction force applied to the area of interest. However, as material is pulled out of the suction tube, the vacuum force will then return to its initial level. These changes may repeat relatively rapidly as material enters and exits the suction tube during the procedure. As a consequence, tissue surrounding the abnormality being removed may be subjected to undue spikes in vacuum force.

In view of the limitations associated with current extraction systems for treating intracranial hematomas and the like, a system permitting the use of a suctioning tube which minimizes insertion trauma while also smoothing pressure spikes at the treatment zone within the brain represents a useful improvement over the current art.

### SUMMARY

Exemplary features of the present disclosure offer advantages and alternatives over the prior art by providing a surgical system adapted for removal of an intracranial abnormality of predetermined size and position. In one exemplary embodiment, the system may include an elongated suction tube including an interior passage extending between an open proximal end and an open distal end. The suction tube comprises an elongated main body portion disposed between the open distal end and a forward end of an enhanced diameter collection cavity. The cross-sectional area of the interior passage varies along the length of the suction tube and is greater within the collection cavity than within the main body portion such that the collection cavity has an internal volume per unit length at least 30% greater than the internal volume per unit length of the main body portion. The total internal volume of the collection cavity preferably exceeds the volume necessary to contain the abnormality. The system further includes an obturator comprising an elongated shaft disposed between a distal tip segment and a proximal handle structure. The distal tip segment may have a rounded closed end to retract tissue, minimizing tissue injury, during the insertion toward the area of interest. The obturator is insertable into the open proximal end of the suction tube such that at least a portion of the distal tip segment extends beyond the elongated suction tube. A positioning probe may be adapted for disposition within the obturator with a portion of the positioning probe extending away from the obturator and adapted to engage a positioning system.

### BRIEF DESCRIPTION OF THE DRAWINGS

FIG. 1 is an elevation perspective view of a system consistent with the present disclosure including a suctioning tube with an enhanced internal diameter collection cavity and an obturator adapted for mating insertion into the proximal end of the suctioning probe in telescoping relation;

FIG. 2 is an elevation assembled view of the system illustrated in FIG. 1 wherein the obturator has been inserted into the suctioning tube causing the distal end of the obturator to project outwardly through the end of the suction tube to define a substantially rounded leading insertion tip;

FIG. 3 is an exploded perspective view illustrating alignment of a positioning probe for insertion into the interior of the obturator to facilitate guided insertion of the obturator and surrounding suctioning tube to the position of interest;

FIG. 4 is an elevation view showing the positioning probe in fully inserted relation within the obturator; and

FIG. 5 is an elevation view illustrating the aligned connection of the suctioning tube to a vacuum line for extraction following withdrawal of the obturator and positioning probe.

Before an exemplary embodiment is explained in detail, it is to be understood that the subject matter is in no way limited in its application or construction to the details and the arrangements of the components set forth in the following description or illustrated in the drawings. Rather, the subject matter is capable of other embodiments and being practiced or being carried out in various ways. Also, it is to be understood that the phraseology and terminology used herein are for purposes of description only and should not be regarded as limiting. The use herein of terms such as “including” and “comprising”, and variations thereof is meant to encompass the items listed thereafter and equivalents thereof as well as additional items and equivalents thereof.

### DETAILED DESCRIPTION OF POTENTIALLY PREFERRED EMBODIMENTS

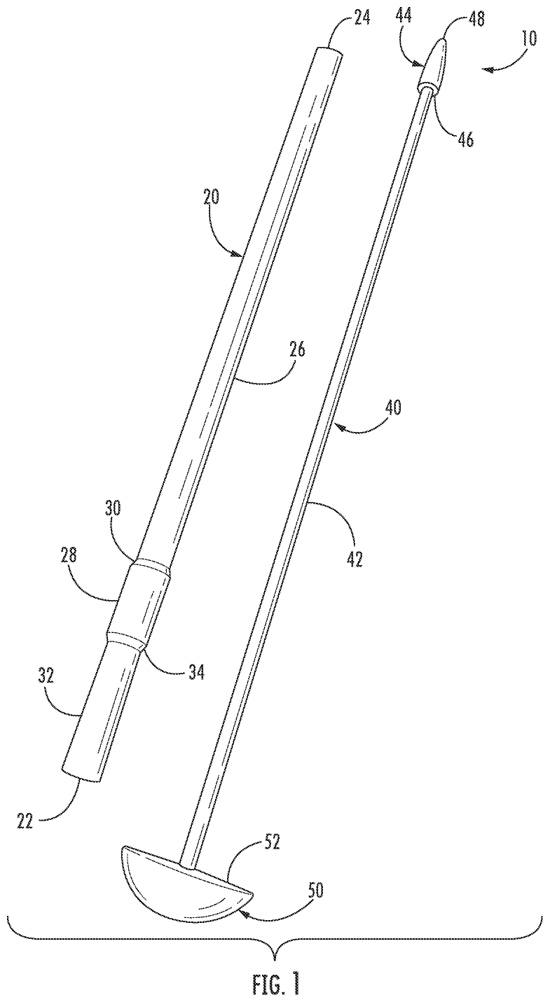

An exemplary embodiment consistent with the present disclosure will now be described in reference to the drawings wherein like elements are designated by like reference numerals in the various views. Referring now to the drawings, FIG. 1 illustrates an exemplary extraction system designated generally as 10 suitable for providing access to, and removal of, intracranial cerebral hematomas and other abnormalities within brain tissue. The extraction apparatus 10 is configured to provide minimally disruptive access to the hematoma or other abnormality followed by suction into an enhanced diameter collection chamber to facilitate more complete removal of abnormalities while maintaining a relatively steady vacuum force at the distal end during the extraction procedure thereby reducing undesired pressure spikes within the zone surrounding the hematoma.

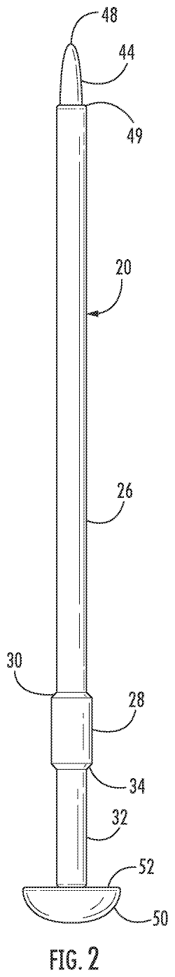

In the illustrated exemplary embodiment, the extraction system 10 comprises a substantially cylindrical hollow suction tube 20 in combination with an obturator 40. As best illustrated in FIG. 2, obturator 40 preferably has an effective outer diameter which is less than the inner diameter of suction tube 20 such that obturator 40 may be selectively inserted into and removed through a rear opening 22 in suction tube 20. Suction tube 20 is hollow along its full length such that there is fluid communication between the rear opening 22 and the distal end 24. Thus, a pressure or vacuum force applied at rear opening 22 will be communicated through the suction tube to the distal end 24. As will be explained further hereinafter, the internal diameter of suction tube 20 may vary along its length to facilitate operation.

Referring first to suction tube 20, in the illustrated exemplary construction, suction tube 20 comprises a main body portion 26 extending rearwardly from distal end 24. Main body portion 26 may have a substantially uniform diameter along its length or may be tapered if desired so as to narrow towards distal end 24. As illustrated, an enhanced diameter collection cavity 28 may be disposed rearward of main body portion 26. In this regard, it is to be understood that the thickness of suction tube 20 may be substantially uniform along its length. Moreover, contour changes at the exterior may be replicated at the interior.

As illustrated, a sloped distal transition zone 30 may be positioned at the intersection between main body portion 26 and collection cavity 28. Such an angled transition may be useful to reduce interference in transporting an extracted hematoma or other abnormality through main body portion 26 and into collection cavity 28. By way of example only, and not limitation, collection cavity 28 may preferably have a length of about 0.75 cm to about 10 cm with an effective internal cross sectional area greater than main body portion 26. Collection cavity 28 may preferably have an internal volume per unit length at least 1.3 times the internal volume per unit length of main body portion 26 (i.e., 30% greater) and most preferably about 1.5-5 times greater than the internal volume per unit length of main body portion 26. The use of this enhanced internal diameter collection cavity provides a collection zone for extracted material and may be helpful to avoid clogging main body portion 26 during an extraction procedure thereby potentially maintaining a more consistent vacuum force at distal end 24. As will be appreciated, if the suction tube becomes unduly obstructed during an extraction procedure, the vacuum force at distal end 24 may be reduced below an effective level. Moreover, when an obstruction is eventually cleared, the vacuum force at the distal end may spike rapidly, which may be undesirable in some instances. Thus, the presence of the increased diameter collection cavity 28 may significantly enhance extraction performance by providing a buffer against clogging and corresponding pressure spikes.

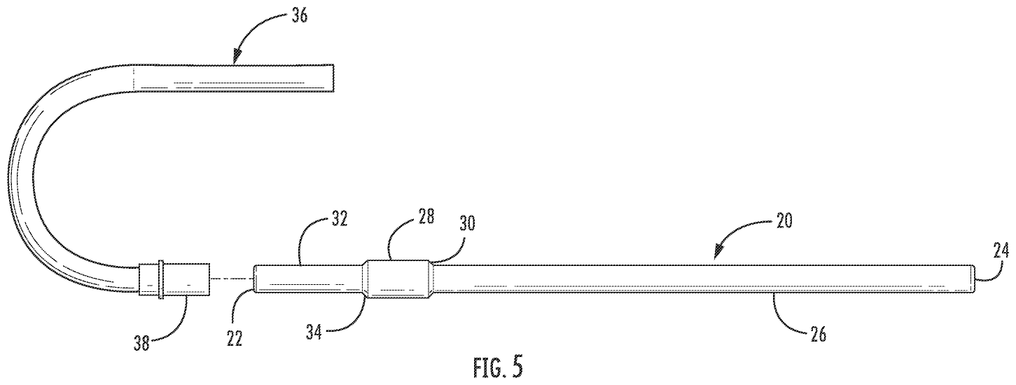

As illustrated, suction tube 20 may further include a proximal nipple segment 32 positioned rearward from collection cavity 28. A sloped proximal transition zone 34 may be positioned at the intersection between proximal nipple segment 32 and collection cavity 28. As best illustrated in FIG. 5, nipple segment 32 may be configured to matedly engage a vacuum line 36 at a connection housing 38. Proximal transition zone 34 facilitates the smooth flow of material out of collection cavity 28 for final removal through vacuum line 36 with a relatively low pressure drop. Additionally, the raised exterior profile at proximal transition zone 34 may be used to ensure proper final positioning of connection housing 38.

As noted previously, extraction apparatus 10 includes an obturator 40 which may be selectively inserted into and removed through rear opening 22 in suction tube 20. As best seen in FIG. 1, obturator 40 may comprise an elongated shaft 42 extending away from a round nose distal cone segment 44. As shown, the distal cone segment 44 may include a base 46 having a diameter greater than elongated shaft 42 to define a shoulder extending radially outwardly at the intersection between elongated shaft 42 and distal cone segment 44. As shown in the illustrated exemplary construction, distal cone segment 44 may have a gradually increasing radius of curvature along its length extending from base 46 to a convex rounded tip 48 such that the radius of curvature is enhanced at convex rounded tip 48. In such constructions, a substantially elliptical or parabolic profile may be particularly desirable. Moreover, rounded tip 48 is preferably substantially smooth with no openings. In accordance with another exemplary construction, distal cone segment 44 may have a profile with substantially straight sides extending away from base with curvature beginning at an elevation above base 46.

As shown, obturator 40 may further include a proximal handle 50 supporting shaft 42. Handle 50 may include an upper platform surface 52 adapted to abut the proximal end of suction tube 20 when obturator 40 is fully inserted (FIG. 2). As shown, in the fully inserted condition, distal cone segment 44 projects outwardly away from the main body portion of suction tube 20. In this condition, rounded tip 48 may be advanced through the brain tissue surrounding the hematoma or other abnormality with minimal disruption while simultaneously urging tissue radially outwardly and establishing a pathway for the trailing suction tube. A rearward sloping beveled edge 49 surrounding distal end 24 may aid in non-traumatic insertion. Likewise, the main body portion 26 may gradually taper from a wide outer diameter adjacent collection cavity 28 to a narrowed diameter at distal end 24 to further facilitate insertion. By way of example only, and not limitation, suction tube 20 and/or obturator 40 may be constructed from biocompatible polymeric material such as polycarbonate, resins or the like. While the suction tube 20 may have a substantially cylindrical cross-section as illustrated, it is likewise contemplated that other cross-sections such as ellipses, ovals and the like may also be used if desired.

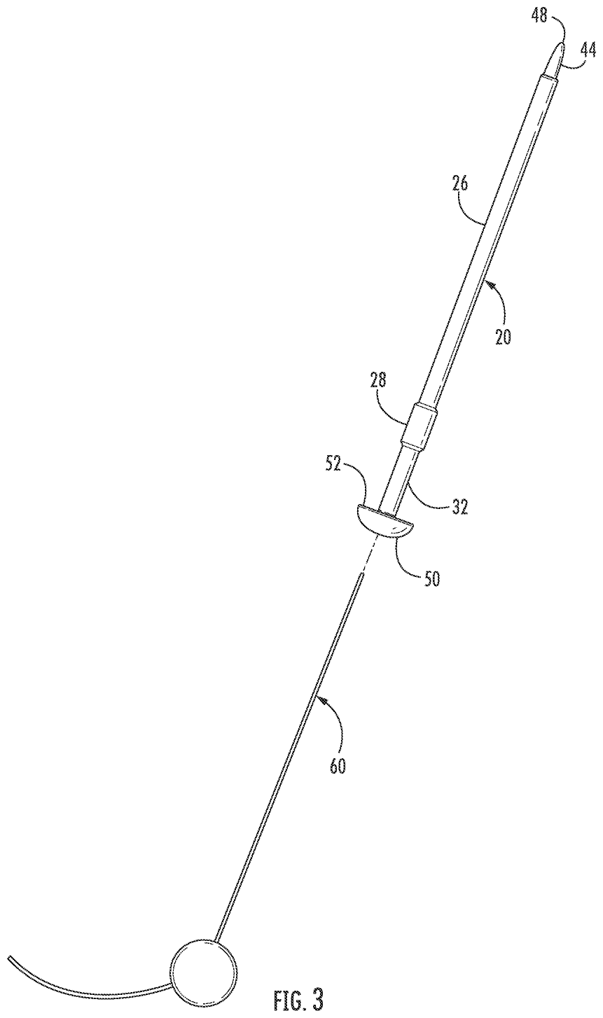

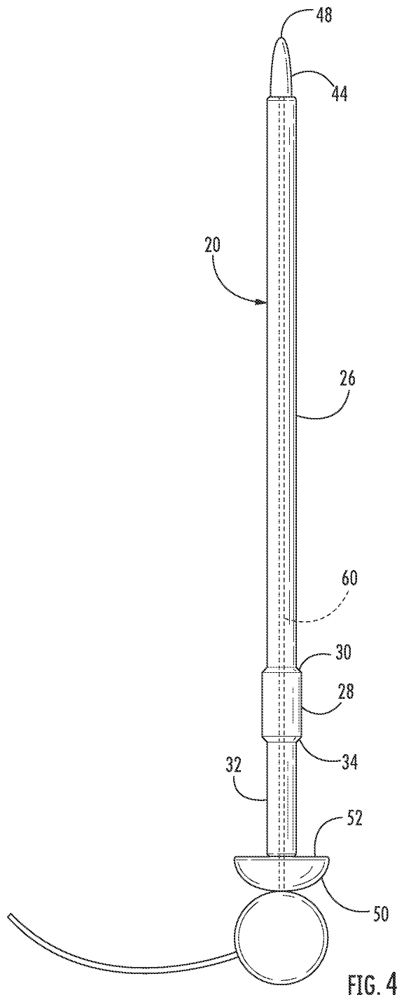

As best seen through joint reference to FIGS. 3 and 4, in accordance with one exemplary practice, obturator 40 may include an interior channel through handle 50 and at least partially along the length of elongated shaft 42 and optionally into the distal cone segment 44 for insertion of a positioning probe 60 operatively linked to an imaging device. With the positioning probe in place, a user may precisely monitor the location of the extraction apparatus as it is advanced to the position of interest. Once the extraction apparatus has reached its desired position within the brain, obturator 40 and positioning probe 60 may then be withdrawn and vacuum line 36 may be connected for the hematoma extraction to commence.

As will be understood, during an extraction procedure, suction force applied through vacuum line 36 is communicated along suction tube 20 to distal end 24 for removal of the hematoma or other abnormality. Although the vacuum force will naturally dissipate to some degree along the length of suction tube 20 due to natural pressure drops, a high vacuum force can be maintained at distal end 24 provided that main body portion 26 does not become excessively clogged. The enhanced diameter collection cavity 28 provides a buffering storage cavity for extracted material which may otherwise clog main body portion 26 and/or nipple segment 32 during extraction. In this regard, it will be understood that the hematoma or other material may not necessarily be suctioned out at a uniform rate. The buffering mechanism provided by collection cavity 28 aids the system in accommodating intermittent enhanced volumes of extracted material without undue clogging thereby permitting the system to operate at a more uniform rate even as the rate of extraction at the distal end may vary.

Enhanced internal diameter collection cavity 28 provides the additional benefit of permitting the surgeon to observe the hematoma mass which has been extracted in real time during the procedure thereby monitoring the extent of the extraction as well as the character of the extracted material. In this regard, collection cavity 28 will be outside the skull during the procedure and may be formed from a substantially transparent polycarbonate or like material to permit content visualization. By choosing suction tube 20 having the proper size collection cavity, substantially the entire hematoma or other abnormality may be captured and retained within collection cavity 28 for observation during the procedure. Thus, it is contemplated that various suction tubes having collection cavities of different sizes may be available for selection and use depending on the size and nature of the mass to be removed as determined by pre-operative imaging.

Preferred embodiments of this disclosure have been described herein, including the best mode known to the inventor for carrying out the disclosure subject matter. Variations of those preferred embodiments may become apparent to those of ordinary skill in the art upon reading the foregoing description. The inventor expects skilled artisans to employ such variations as appropriate and intends for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A surgical system adapted for removal of an intracranial abnormality of predetermined size and position, the system comprising:
an elongated suction tube including an interior passage extending between an open proximal end and an open distal end, the suction tube comprising an elongated main body portion disposed between the open distal end and a forward end of a collection cavity, wherein the cross-sectional area of the interior passage varies along the length of the suction tube and is greater within the collection cavity than within the main body portion such that the collection cavity has an internal volume per unit length at least 30% greater than the internal volume per unit length of the main body portion, the collection cavity defining a buffer zone adapted for collection and visual inspection of extracted abnormality material during suctioning removal, and wherein the total internal volume of the collection cavity exceeds the volume necessary to contain the abnormality;
an obturator comprising an elongated shaft disposed between a distal tip segment and a proximal handle structure, the distal tip segment having a rounded closed end, wherein the obturator is insertable into the open proximal end of the suction tube such that at least a portion of the distal tip segment extends beyond the elongated suction tube; and
at least one positioning probe adapted for disposition within the obturator, a portion of the positioning probe extending away from the obturator and adapted to engage a positioning system.

2. The surgical system as recited in claim 1, wherein the open distal end is surrounded by a rearward sloping beveled edge.

3. The surgical system as recited in claim 1, wherein the suction tube is transparent permitting visual observation of contents within the collection cavity.

4. The surgical system as recited in claim 1, wherein the suction tube further includes a nipple segment disposed between the open proximal end and the collection cavity such that the collection cavity is positioned between the main body portion and the nipple segment.

5. The surgical system as recited in claim 4, wherein the cross-sectional area of the interior passage is greater within the collection cavity than within the nipple segment.

6. The surgical system as recited in claim 1, wherein the collection cavity has an internal volume per unit length in the range 150% to 500% of the internal volume per unit length of the main body portion.

7. The surgical system as recited in claim 1, wherein the obturator distal tip segment comprises a rounded cone having a base of diameter greater than the elongated shaft.

8. The surgical system as recited in claim 1, wherein the obturator includes an interior passage adapted to receive the positioning probe in inserted relation.

9. A surgical system adapted for removal of an intracranial hematoma of predetermined size and position, the system comprising:
An elongated suction tube including an interior passage extending between an open proximal end and an open distal end, the suction tube comprising an elongated main body portion disposed between the open distal end and a forward end of a collection cavity, the suction tube further including a nipple segment disposed between the open proximal end and the collection cavity such that the collection cavity is positioned between the main body portion and the nipple segment, the nipple segment being adapted to operatively engage a vacuum line applying suctioning force through the interior passage, and wherein the cross-sectional area of the interior passage varies along the length of the suction tube and is greater within the collection cavity than within the main body portion such that the collection cavity has an internal volume per unit length at least 30% greater than the internal volume per unit length of the main body portion, the collection cavity defining a buffer zone adapted for collection and visual inspection of extracted abnormality material during suctioning removal, and wherein the total internal volume of the collection cavity exceeds the volume necessary to contain the abnormality;
an obturator comprising an elongated shaft disposed between a distal cone segment and a proximal handle structure, the distal cone segment having a rounded closed tip, wherein the obturator is insertable into the open proximal end of the suction tube such that at least a portion of the distal cone segment extends beyond the elongated suction tube; and
at least one positioning probe adapted for disposition within the obturator, a portion of the positioning probe extending away from the obturator and adapted to engage a positioning system.

10. The surgical system as recited in claim 9, wherein the open distal end is surrounded by a rearward sloping beveled edge.

11. The surgical system as recited in claim 9, wherein the suction tube is transparent permitting visual observation of contents within the collection cavity.

12. The surgical system as recited in claim 9, wherein angled transition zones are disposed between the collection cavity and each of the main body portion and the nipple segment.

13. The surgical system as recited in claim 9, wherein the cross-sectional area of the interior passage is greater within the collection cavity than within the nipple segment.

14. The surgical system as recited in claim 9, wherein the collection cavity has an internal volume per unit length in the range 150% to 500% of the internal volume per unit length of the main body portion.

15. The surgical system as recited in claim 9, wherein the obturator distal tip segment comprises a rounded cone having a base of diameter greater than the elongated shaft.

16. The surgical system as recited in claim 9, wherein the obturator includes an interior passage adapted to receive the positioning probe in inserted relation.
